(19) United States
(12) Patent Application Publication
(10) Pub. No.: US 2025/0299786 A1
(43) Pub. Date: Sep. 25, 2025

(54) METHOD AND APPARATUS FOR ANALYZING BIOMARKER

(71) Applicant: Lunit Lnc., Seoul (KR)

(72) Inventors: Chiyoon OUM, Seoul (KR); Chan-Young OCK, Seoul (KR)

(73) Assignee: Lunit Lnc., Seoul (KR)

(21) Appl. No.: 19/084,997

(22) Filed: Mar. 20, 2025

(30) Foreign Application Priority Data
Mar. 21, 2024 (KR) 10-2024-0038837
Oct. 22, 2024 (KR) 10-2024-0145339

(51) Int. Cl. G16H 10/20

(52) U.S. Cl. CPC G16H 10/20

(57) ABSTRACT

A computing device includes a memory storing at least one program, and a processor configured to perform at least one operation by executing the at least one program, wherein the processor is configured to generate virtual data including information about survival rates of virtual patients included in a first group, based on pre-generated survival data, generate control group data by classifying each of the virtual patients as a responder or a non-responder according to a certain criterion, generate experimental group data based on at least one of medical images and survival data of actual patients included in a second group to which a specific regime has been applied, and output a result of comparison between the control group data and the experimental group data.

### CROSS-REFERENCE TO RELATED APPLICATIONS

This application is based on and claims priority under 35 U.S.C. § 119 to Korean Patent Application No. 10-2024-0038837, filed on Mar. 21, 2024, and Korean Patent Application No. 10-2024-0145339, filed on Oct. 22, 2024, in the Korean Intellectual Property Office, the disclosures of which are incorporated by reference herein in their entirety.

### BACKGROUND

### 1. Field

The disclosure relates to a method and apparatus for analyzing a biomarker.

### 2. Description of the Related Art

To increase a success rate of clinical trials, methods are being developed to search for a patient group with a higher therapeutic response. For example, when responders for a drug are identified among patients through analysis of various medical images (e.g., computed tomography (CT) images, magnetic resonance imaging (MRI) images, and the like) as well as pathology slide images, utility of biomarkers may be confirmed through comparison between the responders and a control group, based on survival data.

However, original data of the clinical trials are not fully disclosed due to data that requires confidentiality, such as personal information of the patients. Accordingly, there are limitations to retrospectively using data collected through prior clinical trials while analyzing biomarkers.

### SUMMARY

Provided are a method and apparatus for analyzing a biomarker, wherein utility of the biomarker is determined by using hypothetical analysis. Also, provided is a computer-readable recording medium having recorded thereon a program for executing the method on a computer. Additional aspects will be set forth in part in the description which follows and, in part, will be apparent from the description, or may be learned by practice of the presented embodiments of the disclosure.

A computing device according to an aspect includes a memory storing at least one program, and a processor configured to perform at least one operation by executing the at least one program, wherein the processor is configured to generate virtual data including information about survival rates of virtual patients included in a first group, based on pre-generated survival data, generate control group data by classifying each of the virtual patients as a responder or a non-responder according to a certain criterion, generate experimental group data based on at least one of medical images and survival data of actual patients included in a second group to which a specific regime has been applied, and output a result of comparison between the control group data and the experimental group data.

A method of analyzing a biomarker, according to another aspect, includes generating virtual data including information about survival rates of virtual patients included in a first group, based on pre-generated survival data, generating control group data by classifying each of the virtual patients as a responder or a non-responder, according to a certain criterion, generating experimental group data based on at least one of medical images and survival data of actual patients included in a second group to which a specific regime has been applied, and outputting a result of comparison between the control group data and the experimental group data.

A computer-readable recording medium, according to another aspect, has recorded thereon a program for executing the method on a computer.

### BRIEF DESCRIPTION OF THE DRAWINGS

FIG. 1 is a diagram for describing an example of analyzing a biomarker, according to an embodiment;

FIG. 2A is a block diagram of an example of a user terminal according to an embodiment;

FIG. 2B is a block diagram of an example of a server according to an embodiment;

FIG. 3 is a flowchart for describing an example of a method of analyzing a biomarker, according to an embodiment;

FIG. 4 is a diagram for describing an example in which a processor generates virtual data, according to an embodiment;

FIG. 5 is a diagram for describing an example in which a processor generates control group data, according to an embodiment;

FIG. 6 is a diagram for describing an example in which a processor generates experimental group data, according to an embodiment;

FIG. 7 is a diagram for describing an example in which a processor generates a result of comparison, according to an embodiment;

FIG. 8 illustrates an example of a user interface (UI) provided to a user when a method of analyzing a biomarker, according to one embodiment, is performed; and

FIG. 9 is a diagram for describing an example of a system for analyzing a biomarker.

### DETAILED DESCRIPTION

Terms used in embodiments have meanings that are obvious to one of ordinary skill in the art, but may have different meanings according to an intention of ordinary skill in the art, precedent cases, or the appearance of new technologies. Also, some terms may be arbitrarily selected by the applicant, and in this case, the meaning of the selected terms will be described in detail in the detailed description. Thus, the terms used herein have to be defined based on the meaning of the terms together with the description throughout the specification.

When a part “includes” or “comprises” an element, unless there is a particular description contrary thereto, the part may further include other elements, not excluding the other elements. In addition, terms such as “unit” and “module” described in the specification denote a unit that processes at least one function or operation, which may be implemented in hardware or software, or implemented in a combination of hardware and software.

Further, the terms including ordinal numbers such as “first”, “second”, and the like used in the specification may be used to describe various components, but the components should not be limited by the terms. The above terms may be used only to distinguish one component from another.

Hereinafter, “medical information” may refer to any medically meaningful information or clinical information of a patient, which may be extracted from a medical image (e.g., a pathology slide image). For example, the medical information may include at least one of an immune phenotype, a genotype, an expressome, a biomarker, tumor purity, information about ribonucleic acid (RNA), a tumor microenvironment, a regime of cancer represented in a pathology slide image, survival information, a treatment response, a treatment outcome, a genetic characteristic, and a medical record.

Also, the medical information may include, but is not limited to, an area, location, or size of a specific tissue (e.g., a cancer tissue or a cancer stromal tissue) and/or a specific cell (e.g., a tumor cell, a lymphocyte cell, a macrophage cell, an endothelial cell, or a fibroblast cell) within a medical image, diagnostic information of cancer, information related to a likelihood of a subject developing cancer, and/or a medical conclusion related to cancer treatment.

In addition, the medical information may include not only a quantitative numerical value that may be obtained from a medical image, but also information obtained by visualizing the numerical value, predicted information based on the numerical value, image information, and statistical information. For example, the medical information may be provided to a user terminal or output through a display device.

Hereinafter, embodiments will be described in detail with reference to accompanying drawings. However, embodiments may be implemented in several different forms and are not limited to those described herein.

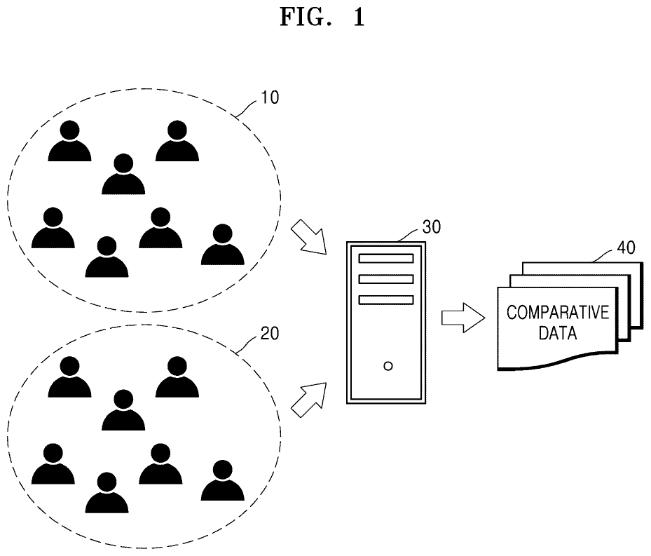

FIG. 1 is a diagram for describing an example of analyzing a biomarker, according to an embodiment.

Referring to FIG. 1, a computing device 30 may output comparative data 40 by using data on a control group 10 (hereinafter, referred to as control group data) and data on an experimental group 20 (hereinafter referred to as experimental group data). For example, the control group 10 may include virtual patients generated based on pre- generated survival data. The experimental group 20 may include actual patients who received specific treatment.

For example, the computing device 30 may confirm a possibility of selecting a patient group through a biomarker (e.g., classifying each of patients as a responder or a non-responder) by applying hypothetical analysis to a result of a pre-performed clinical trial. Specifically, the computing device 30 may generate a virtual control group 10 based on the pre-generated survival data and generate the control group data for the control group 10. Also, the computing device 30 may generate the experimental group data by using the experimental group 20 including actual patients who received a specific regime. The computing device 30 may compare the control group data and the experimental group data to confirm the possibility of selecting a patient group through a biomarker.

For example, when there are a plurality of regimes for specific cancer, information on an effective regime may be provided through the comparative data 40 generated by the computing device 30. In particular, the computing device 30 may provide a guide for selecting a regime through a specific biomarker.

For example, in a case of non-small cell lung cancer (NSCLC), a first regime in which chemotherapy and immunotherapy are combined and a second regime in which only immunotherapy is used may be selected. However, currently, there is no clear guide on which one of the first regime and the second regime is more effective as a treatment for NSCLC, or when is the optimal time to add chemotherapy to a regime.

Specifically, it is difficult to provide a guide on an optimal regime through results of pre-performed clinical trials because not all data of the pre-performed clinical trials is disclosed.

The computing device 30 according to an embodiment generates the control group data by using the survival data derived from pre-performed clinical trials. Also, the computing device 30 generates the experimental group data by using data of actual patients who received a specific treatment. The computing device 30 compares the control group data and the experimental group data to generate the comparative data 40.

Accordingly, a user may select an optimal regime for a specific disease through the comparative data 40. For example, in a case of NSCLC, the user may receive information that the second regime is more effective than the first regime in a patient group exhibiting an inflamed immune phenotype (IIP).

For example, through the comparative data 40, information about which regime is more effective for a patient who exhibits high programmed death-ligand 1 (PD-L1) from among patients with NSCLC, or when is an optimal time to add chemotherapy as a regime may be provided. In other words, the computing device 30 may provide a guide for selecting an optimal regime for each patient through a specific biomarker (e.g., PD-L1 or the like).

Also, the user may obtain information supporting whether a hypothesis established by the user is accurate through the comparative data 40. For example, a hypothesis may be established that “the first regime is more effective for a patient group with non-IIP than for a patient group with IIP, from among patients with NSCLC. In this case, the comparative data 40 may include an effect of the first regime and an effect of the second regime in a patient group with IIP. The comparative data 40 may also include an effect of the first regime and an effect of the second regime in a patient group with non-IIP. Accordingly, the user may determine whether the hypothesis he/she has established is correct through the comparative data 40.

The biomarker may be a biomarker identified through a machine learning model. For example, in a case of NSCLC, exhibition of PD-L1 identified from pathology slide images through a machine learning model may be a biomarker. The machine learning model may use pre-generated medical information to identify a biomarker associated with a specific disease. For example, the biomarker may include, but are not limited to, PD-L1, epidermal growth factor receptor (EGFR), ductile carcinoma in situ (DCIS), anaplastic lymphoma kinase (ALK), endoplasmic reticulum (ER), human epidermal growth factor receptor 2 (HER2), and initialism of vascular endothelial growth factor (VEGF).

For example, the computing device 30 may be a user terminal or a server.

The user terminal may be an electronic device including a display device and a device for receiving a user input (e.g., a keyboard, a mouse, or the like), and including a memory and a processor. The display device may be implemented as a touch screen and perform a function of receiving a user input. For example, the user terminal may include, but are not limited to, a notebook personal computer (PC), a desktop PC, a laptop PC, a tablet computer, a smartphone, or the like.

The server may be a device configured to communicate with an external device (e.g., the user terminal). For example, the server may be a device storing various types of data, including medical information and information about a machine learning model. Alternatively, the server may be an electronic device that includes memory and a processor and has self-arithmetic capability. For example, the server may be, but is not limited to, a cloud server.

The computing device 30 may analyze a pathology slide image to identify biological factors (e.g., cancer cells, immune cells, or cancer regions) or a biomarker represented in the pathology slide image. Such biological factor or biomarker may be used for histological diagnosis of a disease, prediction of disease prognosis, and determination of a treatment direction for a disease.

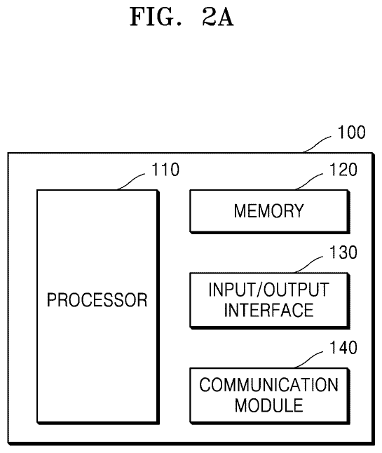

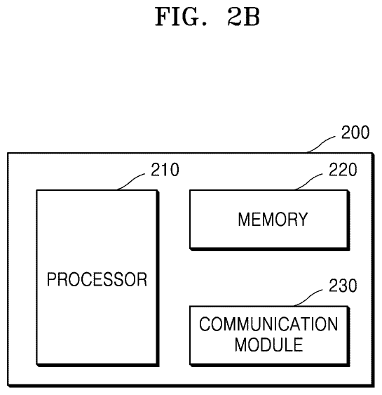

Hereinafter, an example in which the computing device 30 analyzes a biomarker will be described with reference to FIGS. 2A to 8.

As described above, the computing device 30 may be a user terminal or a server. Accordingly, hereinafter, operations performed by the computing device 30 may be performed by a user terminal or a server. Alternatively, hereinafter, some of the operations performed by the computing device 30 may be performed by the user terminal and the remaining operations may be performed by the server.

Hereinafter, examples of a user terminal and a server will be described with reference to FIGS. 2A and 2B.

FIG. 2A is a block diagram of an example of a user terminal 100 according to an embodiment.

Referring to FIG. 2A, the user terminal 100 includes a processor 110, a memory 120, an input/output interface 130, and a communication module 140. For convenience of description, only components related to the disclosure are illustrated in FIG. 2A. Accordingly, in addition to the components illustrated in FIG. 2A, other general-purpose components may be further included in the user terminal 100. In addition, it would be obvious to one of ordinary skill in the art that the processor 110, the memory 120, the input/output interface 130, and the communication module 140 illustrated in FIG. 2A may be implemented as independent devices.

The processor 110 may be configured to process a command of a computer program by performing basic arithmetic, logic, and input/output operations. Here, the command may be provided from memory 120 or an external device (e.g., a server 200 or the like). Also, the processor 110 may generally control operations of other components included in the user terminal 100.

The processor 110 generates virtual data including information about survival rates of virtual patients included in a first group, based on pre-generated survival data. For example, the processor 110 may generate data on at least one of progression-free survival and overall survival of each of the virtual patients by using the pre-generated survival data.

For example, the processor 110 may obtain a Kaplan-Meier curve for at least one of the progression-free survival and overall survival. Also, the processor 110 may select a certain number of points on the Kaplan-Meier curve. Then, the processor 110 may generate data on at least one of the progression-free survival and the overall survival of each of the virtual patients by using coordinate values corresponding to the points.

The processor 110 generates control group data by classifying each of virtual patients as a responder or a non-responder according to a certain criterion. For example, the processor 110 may set a proportion of responders and at least one parameter value, based on a hypothesis to be verified. The processor 110 may generate at least one set in which the virtual patients are classified as responders or non-responders, based on the set proportion and the parameter value. In other words, the processor 110 may additionally generate information indicating whether each patient is a responder or a non-responder, in addition to the data on the progression-free survival or overall survival of the virtual patients. Here, the at least one parameter value may include at least one of a hazard ratio of the progression-free survival and a hazard ratio of the overall survival.

For example, the processor 110 may determine the proportion of responders based on information about a regime corresponding to a drug that is basis for the pre-generated survival data. The processor 110 may generate the at least one set such that at least one parameter value is satisfied.

The processor 110 generates experimental group data based on medical images and survival data of actual patients included in a second group who received a specific treatment. The processor 110 may directly obtain the progression-free survival or overall survival of patients as the survival data or may predict the progression-free survival or overall survival of patients from a graph representing at least one of the progression-free survival or overall survival. For example, the processor 110 may predict the progression-free survival or overall survival of patients from the Kaplan-Meier curve. The processor 110 may generate the experimental group data including data on the progression-free survival or overall survival of patients, together with information about classifying patients included in the second group as responders or non-responders, based on biomarkers identified from the medical images.

The processor 110 outputs a result of comparison between the control group data and the experimental group data. For example, the processor 110 may perform a plurality of simulations by comparing at least one control group data set and at least one experimental group data set, respectively. The processor 110 may perform the plurality of simulations by repeatedly performing analysis of comparing various combinations of a plurality of control group data sets included in the control group data and a plurality of experimental group data sets included in the experimental group data.

The processor 110 may generate and output the comparative data 40 obtained by summarizing results of the plurality of simulations. Here, the control group data and the experimental group data are in the form of a table in which the information about whether the patients are responders or non-responders is combined with the data on at least one of the progression-free survival and overall survival of the patients. Data may be compared by performing a plurality of simulations according to a hypothesis set by a user, and a result of comparative analysis may include at least one of the number of comparative analyses in which a significant difference was found between control group and experimental group (or a proportion (%) of the number of comparative analyses in which significant results were derived compared to the total number of comparative analyses), a hazard ratio of progression-free survival, and a hazard ratio of overall survival.

The processor 110 may be implemented as an array of a plurality of logic gates, or in a combination of a general-purpose microprocessor and a memory storing a program executable by the general-purpose microprocessor. For example, the processor 110 may include a general-purpose processor, a central processing unit (CPU), a microprocessor, a digital signal processor (DSP), a controller, a microcontroller, a state machine, and the like. In some environments, the processor 110 may include to an application-specific semiconductor (ASIC), a programmable logic device (PLD), a field programmable gate array (FPGA), or the like. For example, the processor 110 may refer to a combination of processing devices, such as a combination of a DSP and a microprocessor, a combination of a plurality of microprocessors, a combination of one or more microprocessors coupled with a DSP core, or a combination of any other such components.

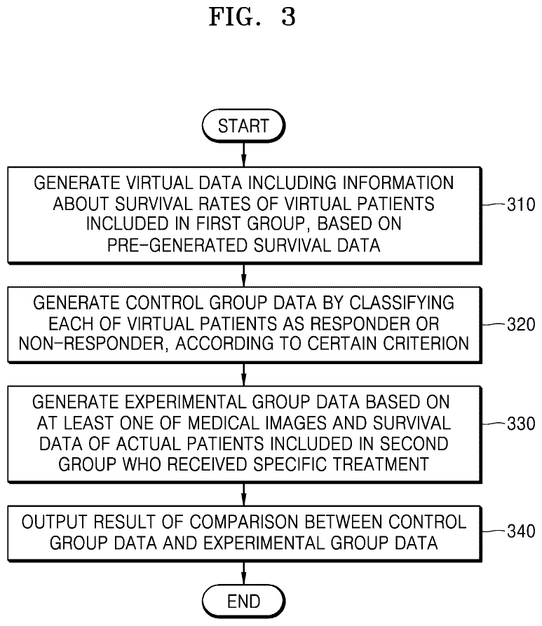

The memory 120 may include any non-transitory computer-readable recording medium. For example, the memory 120 may include a permanent mass storage device, such as random access memory (RAM), read-only memory (ROM), disk drive, solid state drive (SSD), or flash memory. In another example, the permanent mass storage device, such as ROM, SSD, flash memory, or disk drive, may be a separate permanent storage device distinguished from a memory. The memory 120 may store an operating system (OS) and at least one program code (e.g., code for the processor 110 to perform operations described below with reference to FIGS. 3 to 8).

Such software components may be loaded from a computer-readable recording medium separate from the memory 120. The separate computer-readable recording medium may be a recording medium that may be directly connected to the user terminal 100, and for example, may include a computer-readable recording medium such as floppy drive, disk, tape, DVD/CD-ROM drive, or memory card. The software components may be loaded into the memory 120 through the communication module 140, instead of the computer-readable recording medium. For example, at least one program may be loaded into the memory 120, based on a computer program (e.g., a computer program for the processor 110 to perform operations described below with reference to FIGS. 3 to 8) installed by files provided by developers or by a file distribution system for distributing an installation file of an application through the communication module 140.

The input/output interface 130 may be a unit for interfacing with a device (e.g., a keyboard, a mouse, or the like) for input and/or output, which may be connected to or included in the user terminal 100. In FIG. 2A, the input/output interface 130 is illustrated as an element configured separately from the processor 110, but is not limited thereto, and the input/output interface 130 may be included in the processor 110.

The communication module 140 may provide a configuration or function enabling the server 200 and the user terminal 100 to communicate with each other. In addition, the communication module 140 may provide a configuration or function enabling the user terminal 100 to communicate with other external devices. For example, control signals, commands, data, or the like provided under control by the processor 110 may be transmitted to the server 200 and/or an external device through the communication module 140 and the network.

Although not shown in FIG. 2A, the user terminal 100 may further include a display device. Alternatively, the user terminal 100 may be connected to an independent display device via wired or wireless communication to transmit and receive data between the user terminal 100 and the display device. For example, a report including the pathology slide images, analysis information of the pathology slide images, the medical information, additional information based on the medical information, and the comparative data 40 may be provided to the user through the display device.

FIG. 2B is a block diagram of an example of the server 200 according to an embodiment.

Referring to FIG. 2B, the server 200 include a processor 210, a memory 220, and a communication module 230. For convenience of description, only components related to the disclosure are illustrated in FIG. 2B. Accordingly, in addition to the components illustrated in FIG. 2B, other general-purpose components may be further included in the server 200. In addition, it would be obvious to one of ordinary skill in the art that the processor 210, the memory 220, and the communication module 230 illustrated in FIG. 2B may be implemented as independent devices.

The processor 210 may obtain a pathology slide image from at least one of the internal memory 220, the user terminal 100, and another external device. The processor 210 may generate the virtual data including the information about the survival rates of the virtual patients included in the first group, based on the pre-generated survival data, generate the control group data by classifying each of the virtual patients as a responder or a non-responder, based on the certain criterion, generate the experimental group data based on at least one of the medical images and the survival data of the actual patients included in the second group who received a specific treatment, or output the result of comparison between the control group data and the experimental group data. Alternatively, the processor 210 may transmit data (or a report) including the result of comparison to the user terminal 100.

In other words, at least one of the operations of the processor 110 described above with reference to FIG. 2A may be performed by the processor 210. In this case, the user terminal 100 may output information transmitted from the server 200 through the display device.

Because an implementation example of the processor 210 is the same as an implementation example of the processor 110 described above with reference to FIG. 2A, a detailed description will not be provided.

Various types of data, such as data generated according to an operation of the processor 210, may be stored in the memory 220. The memory 220 may also store an operating system (OS) and at least one program (e.g., a program required for the processor 210 to operate).

Because an implementation example of the memory 220 is the same as an implementation example of the memory 120 described above with reference to FIG. 2A, a detailed description will not be provided.

The communication module 230 may provide a configuration or function enabling the server 200 and the user terminal 100 to communicate with each other. In addition, the communication module 230 may provide a configuration or function enabling the server 200 to communicate with other external devices. For example, control signals, commands, data, or the like provided under control by the processor 210 may be transmitted to the user terminal 100 and/or an external device through the communication module 230 and the network.

FIG. 3 is a flowchart for describing an example of a method of analyzing a biomarker, according to an embodiment.

The method illustrated in FIG. 3 includes operations processed in time series by the computing device 30 or the processor 110 or 210 illustrated in FIGS. 1 to 2b. Accordingly, even if omitted below, the details described above with respect to the computing device 30 or the processor 110 or 210 may also be applied to the method of FIG. 3.

Also, as described above with reference to FIG. 2B, hereinafter, at least one of operations performed by the processor 110 may be performed by the processor 210.

In operation 310, the processor 110 generates the virtual data including the information about the survival rates of the virtual patients included in the first group, based on the pre-generated survival data.

For example, the processor 110 may generate the data on at least one of the progression-free survival and the overall survival of each of the virtual patients by using the pre-generated survival data. Here, the survival data may include data representing changes in survival rates for specific patients according to time.

For example, the survival data may be provided in various forms, such as a graph, a drawing, a diagram, a table, and a document. Also, the survival data may include, but is not limited to, a Kaplan-Meier curve.

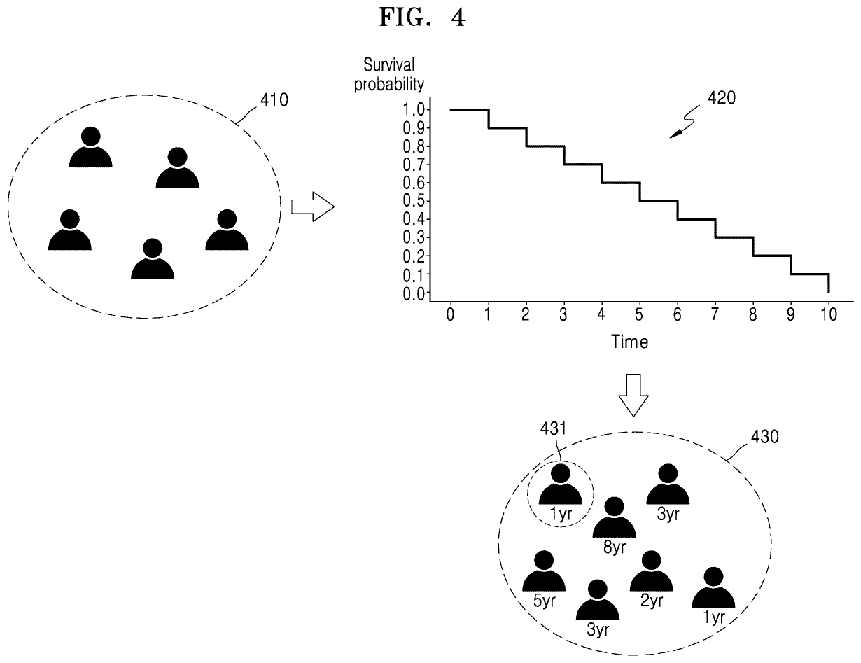

The pre-generated survival data may be obtained from research reports, papers, research data databases, and the like, which include survival analysis results from prior clinical trials. Hereinafter, an example in which the processor 110 generates the virtual data will be described with reference to FIG. 4.

FIG. 4 is a diagram for describing the example in which the processor 110 generates the virtual data, according to an embodiment.

Referring to FIG. 4, the processor 110 generates virtual data by using pre-generated survival data 420. For example, the virtual data may include data on at least one of progression-free survival and overall survival of each of virtual patients 430. Here, the pre-generated survival data 420 may be data representing changes in survival rates of specific patients 410. Hereinafter, it is assumed that the pre-generated survival data 420 is a Kaplan-Meier curve.

First, the processor 110 obtains a Kaplan-Meier curve 420. For example, the processor 110 may obtain the Kaplan-Meier curve 420 of progression-free survival or overall survival described in a paper, based on a clinical trial that may be used as a comparative group depending on a purpose of a user.

The processor 110 selects a certain number of points on the Kaplan-Meier curve 420. For example, the number of points may be pre-set or set by the user.

Then, the processor 110 generates data on at least one of progression-free survival and overall survival of each of the virtual patients 430 by using coordinate values corresponding to the points. For example, the processor 110 may pre-process data corresponding to the selected points to estimate a risk of occurrence of an event at the selected points. In addition, the processor 110 may reconstruct individual patient data in which occurrence of an event and a time elapsed until the occurrence (time-to-event data) are defined for each patient, through a result of the estimation and the number of patients for whom an event did not occur at each pre-set time point. For example, an event may include an event that may indicate progress of treatment on a patient, such as the patient's death, relapse, or cure.

Accordingly, the processor 110 may generate data (i.e., the virtual data) on the progression-free survival or overall survival of virtual patients 430 through the Kaplan-Meier curve 420. For example, progression-free survival or overall survival may be set for each patient 431 of the virtual patients 430. At this time, one of the progression-free survival and the overall survival may be selected depending on the hypothesis set by the user.

Referring back to FIG. 3, in operation 320, the processor 110 generates the control group data by classifying each of the virtual patients as a responder or a non-responder, according to the certain criterion.

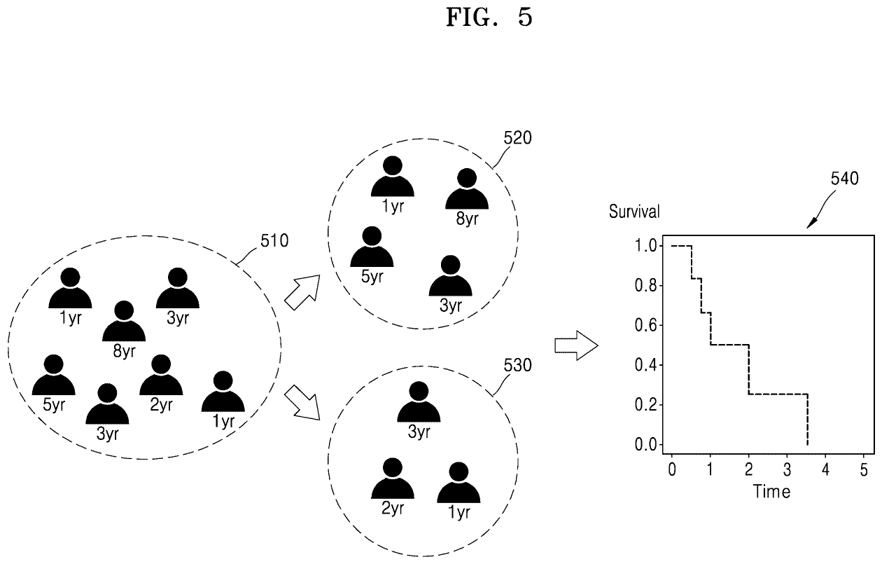

The processor 110 may generate the control group data by assigning the virtual patients 430 as responders or non-responders according to the certain criterion. For example, the processor 110 may, randomly or according to the certain criterion, assign each of the virtual patients 430 as a responder or a non-responder. Here, the certain criterion may be set based on the hypothesis set by the user. Hereinafter, an example in which the processor 110 generates the control group data will be described with reference to FIG. 5.

FIG. 5 is a diagram for describing an example in which the processor 110 generates the control group data, according to an embodiment.

Referring to FIG. 5, the processor 110 classifies virtual patients 510 included in a first group as responders 520 or non-responders 530, according to a certain criterion. Here, the certain criterion includes a proportion of responders and at least one parameter value. For example, the processor 110 assigns response statuses of virtual patients such that a response status proportion and one parameter value are satisfied based on results of prior clinical trials or a pre-set default value, according to a hypothesis set by the user.

The virtual patients 510 of FIG. 5 may be the same as the virtual patients 430 of FIG. 4. The processor 110 may generate control group data 540 including results of classifying patients included in a control group as responders or non-responders, and data on progression-free survival or overall survival of the patients.

In detail, the processor 110 may determine a proportion of the responders 520 among the virtual patients 510, according to the at least one parameter value set based on the hypothesis. Then, the processor 110 may generate at least one set in which each of the virtual patients 510 is classified as a responder 520 or the non-responder 530, based on the determined proportion. Here, the parameter value includes at least one of a hazard ratio of the progression-free survival and a hazard ratio of the overall survival.

In other words, the processor 110 may set an appropriate parameter value based on the results of prior clinical trials or the pre-set default value, according to the hypothesis set by the user. The processor 110 may arbitrarily designate some of the virtual patients 510 as the responders 520 for a biomarker, according to the set parameter value.

At this time, the processor 110 determines the proportion of responders 520 among the virtual patients 510, based on information about a regime corresponding to a drug that is basis of the pre-generated survival data 420. The processor 110 generates at least one set by using the data of the virtual patients 510 so that the parameter value is satisfied. In other words, the control group data 540 may be include at least one set.

For example, it is assumed that a regime corresponding to a drug that is basis of study (e.g., a clinical trial) in which the survival data 420 was generated is Regime A. Then, it is assumed that a proportion of responders among a patient group participating in the study in which the survival data 420 was generated is 40%. Also, it is assumed that there are a total of 1,000 virtual patients 510 included in the first group. In this case, the processor 110 may assign some of the virtual patients 510 as the responders 520 so that there are 400 responders 520.

At this time, the processor 110 may generate at least one set by using data on the virtual patients 510 so that a parameter value (i.e., a hazard ratio of progression-free survival or a hazard ratio of overall survival) is satisfied. In the above example, the processor 110 may determine criteria for generating a set as follows: i) data included in one set will include data on 400 responders 520, and ii) data included in one set will satisfy a hazard ratio of progression-free survival or a hazard ratio of overall survival for each responder 520 or each non-responder 530. Then, the processor 110 may generate b sets according to the above criteria. At this time, b may be determined to be any one of various numbers, such as 1, 100, 1,000, and 10,000, depending on a purpose of hypothetical analysis.

Accordingly, the control group data 540 may be include b sets. As described above with reference to FIG. 4, the data on the progression-free survival or the overall survival may be generated for each of the virtual patients 510. Accordingly, the processor 110 may generate the control group data 540 including b sets by using the data of the virtual patients 510.

As described above with reference to FIG. 5, at least one set is generated in which some of the virtual patients 510 included in the first group are randomly set as responders. At this time, as a criterion for generating the set, a hazard ratio of progression-free survival or a hazard ratio of overall survival for each responder depending on a proportion of responders for a biomarker or whether a patient is a responder may be considered. Here, the proportion of responders or whether a patient is a responder may be derived from results of the study in which the survival data 420 was generated.

Referring back to FIG. 3, in operation 330, the processor 110 generates the experimental group data based on at least one of the medical images and the survival data of the actual patients included in the second group to which a specific regime is applied.

For example, the processor 110 may generate at least one set in which the patients included in the second group are classified as responders or non-responders, based on biomarkers identified from the medical images. In other words, the experimental group data may include at least one set.

When the user requests generation of experimental group data 640 by providing only medical images, the processor 110 may arbitrarily generate the experimental group data 640 based on a result of survival analysis performed by the user (e.g., a graph image showing survival rates of patients, such as a Kaplan-Meier graph).

As described above with reference to FIGS. 4 and 5, the control group data is generated based on the first group including the virtual patients. Here, the virtual patient refers to a patient who does not actually exist, but is fabricated by using pre-generated survival data.

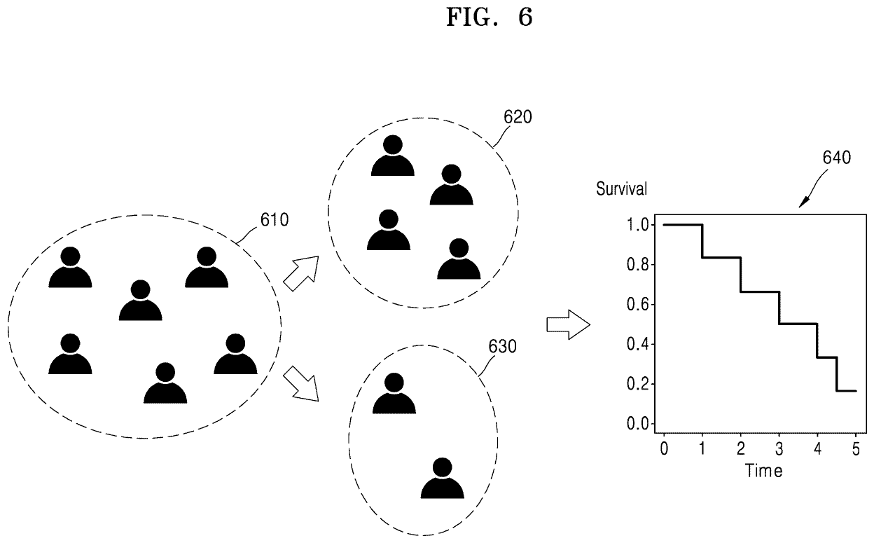

Meanwhile, the experimental group data is generated based on the second group including the actual patients. Here, an actual patient refers to a patient to whom a specific regime is applied (i.e., a specific treatment is performed). In other words, the control group data refers to data about virtual patients fabricated based on actual data (i.e., pre-generated survival data), and the experimental group data refers to data generated based on patients who actually received treatment. Hereinafter, an example in which the processor 110 generates the experimental group data will be described with reference to FIG. 6.

FIG. 6 is a diagram for describing an example in which the processor 110 generates the experimental group data, according to an embodiment.

Referring to FIG. 6, the processor 110 classifies actual patients 610 included in a second group as responders 620 or non-responders 630. Here, the actual patients 610 refer to patients to whom a specific regime is applied (i.e., a treatment is performed). Then, the processor 110 generates the experimental group data 640 based on a result of the classification (i.e., a result of classifying patients included in an experimental group as responders or non-responders). For example, the experimental group data 640 may include information about progression-free survival or overall survival.

The processor 110 may generate at least one set by classifying the actual patients 610 as the responders 620 or the non-responders 630, based on biomarkers identified from medical images of each of the actual patients 610. In other words, the experimental group data 640 may be include at least one set.

For example, the processor 110 may generate a sets of the experimental group data 640 from the medical images and the survival data of the patients 610 who received a specific treatment. Here, the experimental group data 640 may also include a result of determining whether each patient 610 is a responder or a non-responder, based on a specific biomarker derived from the medical images of the patients 610. Also, as described above with reference to FIG. 5, a may be determined to be any one of various numbers, such as 1, 100, 1,000, and 10,000, depending on a purpose of hypothetical analysis.

For example, when both the medical images and the survival data (i.e., progression-free survival or overall survival) of the patients 610 are secured, the experimental group data 640 may include one data set. On the other hand, when only the medical images of the patients 610 are secured, the processor 110 may predict the progression-free survival or the overall survival of the patients 610 from a graph representing at least one of the progression-free survival or the overall survival. In this case, the processor 110 may generate the experimental group data 640 including a plurality of data sets that satisfy a parameter value (i.e., a hazard ratio of progression-free survival or a hazard ratio of overall survival).

Referring back to FIG. 3, in operation 340, the processor 110 outputs the result of comparison between the control group data and the experimental group data.

For example, the processor 110 may perform a plurality of simulations by comparing various combinations of the at least one set included in the control group data and the at least one set included in the experimental group data. Accordingly, the processor 110 may generate the result of comparison. For example, the result of comparison may include a comparison of survival rates of responders selected by a biomarker, for each regime, when hypothetical analysis is performed through the plurality of simulations.

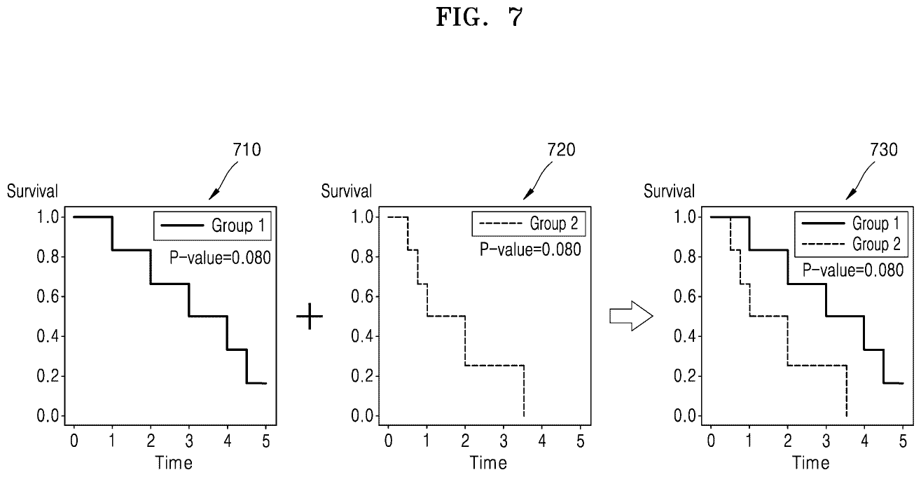

The result of comparison may be output in the form of a report. For example, the report may correspond to data in various forms, such as a graph, a drawing, a diagram, a table, and a document. Hereinafter, an example in which the processor 110 generates a plurality of results of comparison between the control group data and the experimental group data will be described with reference to FIG. 7.

FIG. 7 is a diagram for describing an example in which the processor 110 generates the result of comparison, according to an embodiment.

Referring to FIG. 7, the processor 110 may generate a plurality of results 730 of comparisons between control group data 710 and experimental group data 720. For example, it is assumed that the control group data 710 includes b sets and the experimental group data 720 includes a sets. In this case, the processor 110 may generate a×b results 730 of comparisons.

For example, the results 730 of comparisons may include a graph showing results of survival analysis according to a hazard ratio of progression-free survival or a hazard ratio of overall survival, and a certain confidence interval (e.g., a confidence interval of 95%). In addition, the results 730 of comparisons may include a table or graph showing a×b results of comparisons for each of responders and non-responders. At this time, the table or graph may display the number of comparative analyses in which significant differences were found among the a×b results of comparisons (or a proportion (%) of the number of comparative analyses in which significant results were derived compared to the number of all comparative analyses).

The results 730 of comparisons may be used to explore or verify validity of a biomarker identified by a machine learning model. For example, it is assumed that there was no significant difference in prognoses of patients depending on a regime of an experimental group and a regime of a control group, in prior studies (e.g., clinical trials). On the other hand, it is assumed that a significant difference was confirmed between two regimes (i.e., a regime of an experimental group and a regime of a control group) depending on whether a patient is a responder or a non-responder, or that tendencies of results of comparisons vary depending on whether a patient is a responder, in the results 730 of comparisons. In this case, a possibility of selecting a responder through a biomarker may be confirmed by the results 730 of comparisons. In other words, the results 730 of comparisons through a plurality of simulations may serve as a basis for exploring and evaluating a biomarker that may more clearly confirm a prognosis of a patient.

Thus, according to various embodiments of the disclosure, it is possible to derive a large number of results of comparisons that are physically impossible for a person to calculate. In addition, by enabling analyses of the large number of results of comparisons, reliable results may be derived for hypothesis testing related to a regime.

Operations of the processor 110 described above with reference to FIGS. 1 to 7 will be described below with an example.

For NSCLC, immunotherapy is chosen as a primary regime, but there is no clear guide on what an optimal regime, especially when is an appropriate time to add chemotherapy. At this time, a user may establish a hypothesis that a combination of immunotherapy and chemotherapy (hereinafter, referred to as a “first regime”) results in a better prognosis for a patient group exhibiting non-IIP and that applying only immunotherapy (hereinafter, referred to as a “second regime”) results in a better prognosis for a patient group exhibiting IIP.

The processor 110 may generate virtual data based on pre-generated survival data (e.g., a Kaplan-Meier curve). For example, the processor 110 may analyze survival data from a prior study of 205 patients to whom the first regime was applied and survival data from a prior study of 453 patients to whom the second regime was applied. Then, the processor 110 may assume virtual patients and generate virtual data including information about survival rates of the virtual patients.

The processor 110 may classify the virtual patients as responders or non-responders, based on a certain criterion. For example, the processor 110 may classify, among the virtual patients, virtual patients with IIP to be 57% and virtual patients with non-IIP to be 43%. Here, the above-mentioned numerical values (i.e., 57% and 43%) may be values obtained through inference from results of prior experiments. The processor 110 may generate control group data including 100 sets, based on results of classifying the virtual patients.

Also, the processor 110 may generate experimental group data based on data of actual patients to whom a specific regime (i.e., the first regime or the second regime) has been applied. For example, the processor 110 may predict an immune phenotype of patients by analyzing pathology slide images of the actual patients. The processor 110 may generate 100 sets that satisfy a hazard ratio of non-IIP to IIP to be 0.81 and 0.61 (±0.025), while maintaining a proportion of patients exhibiting IIP, based on the predicted immune phenotype. In other words, the processor 110 may generate 100 sets by assigning progression-free survival or overall survival to the patients so that the hazard ratio within 0.61 (±0.025) is calculated. For example, 100 sets in which of immune activation and survival data of each patient are variously combined may be generated. The processor 110 may generate the experimental group data including 100 sets.

The processor 110 may analyze 10,000 cases in which the control group data including 100 sets and the experimental group data including 100 sets are combined. Then, the processor 110 may output analysis results. For example, the analysis results may be used as a basis to support whether the hypothesis established by the user is correct.

For example, it is assumed that the analysis results show that a median survival rate of patients to whom the first regime was applied was higher than a median survival rate of patients to whom the second regime was applied (a hazard ratio 0.73, 95% confidence interval, and p=0.045). In this case, 6,917 out of 10,000 analyses may be determined to support the accuracy of the hypothesis. In other words, the hypothetical analysis described above suggests that the patients with the non-IIP exhibit a better effect from the first regime than the patients with the IIP, at a proportion of 69.17%. Accordingly, based on the analysis results, the necessity of chemotherapy in the treatment of NSCLC may be evaluated through analysis of the immune phenotype. Also, the analysis results may be provided as guidelines for customized treatment for each patient.

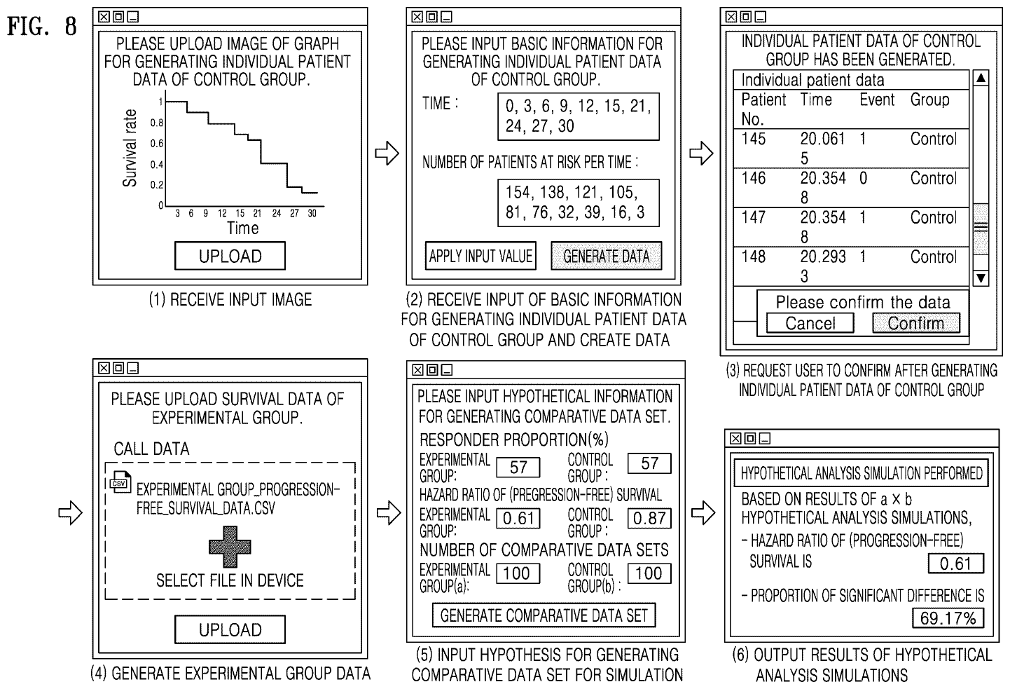

FIG. 8 illustrates an example of a user interface (UI) provided to a user when a method of analyzing a biomarker, according to one embodiment, is performed.

Referring to FIG. 8, in operation (1), a screen including a UI for uploading pre-generated survival data may be output. For example, an image of a Kaplan-Meier curve may be uploaded as the pre-generated survival data.

In operation (2), a UI for inputting basic information for generating virtual data may be output. Based on the basic information input through the UI, survival data for each virtual patient may be generated. The basic information may include a time and the number of patients at risk per time. A Kaplan-Meier graph may include a table at the bottom of the graph, showing the number of patients at per time, and the number of patients at risk per time recorded in the table may be input as the basic information.

In operation (3), individual patient data of a control group, which is used to generate control group data, may be generated and output based on data obtained in operation (1) and operation (2). A UI for a user to confirm the generated individual patient data of the control group may be output.

When the user confirms the control group data generated for the virtual patients, a screen including a UI for uploading data on which an actual experiment has been performed may be output in operation (4). The data uploaded in operation (4) may include a list of patients on whom experiments were performed, a treatment response (or immune phenotype) of each patient derived from analysis of a medical image of the patient, and survival information of each patient.

In operation (5), a UI for inputting a hypothesis used to compare control group data and experimental group data may be output. For example, the user may input a responder proportion, a hazard ratio of progression-free survival, and the number of comparative data sets, based on the hypothesis to be verified. After the user inputs the hypothesis and clicks a “Generate comparative data” button, the processor 110 may perform a simulation for hypothetical analysis and output a result of the simulation.

Based on the hypothesis input by the user, the control group data including a plurality of data sets may be generated from the individual patient data of the control group output in operation (3), and the experimental group data including a plurality of data sets may be generated from the data input in operation (4). For example, the processor 110 may perform a plurality of simulations by comparing various combinations of the plurality of data sets included in the control group data and the plurality of data set included in the experimental group data, and output results of comparisons.

Operation (5) of FIG. 8 illustrates a case where the responder proportion, the hazard ratio of survival, and the number of comparative data sets are input as the hypothesis, but the disclosure is not limited thereto, and the simulations may be performed by adding other parameters as the hypothesis in addition to the hazard ratio. For example, information such as a gender, an age group, a race, genetics, a smoking status, and the like may be used as the other parameters.

In operation (6), the hazard ratio of progression-free survival and a proportion of a significant difference in the survival rate between the control group and the experimental group may be output as analysis results.

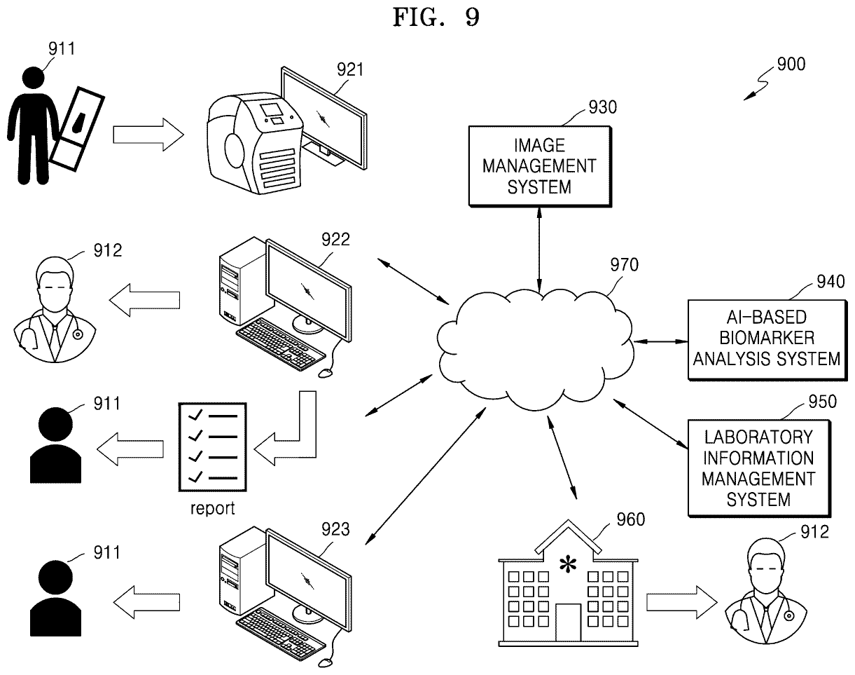

FIG. 9 is a diagram for describing an example of a system 900 for analyzing a biomarker.

Referring to FIG. 9, the system 900 is an example of a system and network for analyzing a biomarker by using a machine learning model.

According to various embodiments of the disclosure, the method described above with reference to FIGS. 2A to 7 may be performed by at least one or a combination of user terminals 922 and 923, an image management system 930, an AI-based biomarker analysis system 940, a laboratory information management system 950, and a hospital or laboratory server 960.

A scanner 921 may obtain a digitized image from a tissue sample slide generated by using a tissue sample of a subject 911. For example, the scanner 921, the user terminals 922 and 923, the image management system 930, the AI-based biomarker analysis system 940, the laboratory information management system 950, and/or the hospital or laboratory server 960 may each be connected to a network 970 such as the Internet through one or more computers, servers, and/or mobile devices, or may communicate with a user 912 through one or more computers and/or mobile devices.

The user terminals 922 and 923, the image management system 930, the AI-based biomarker analysis system 940, the laboratory information management system 950, and/or the hospital or laboratory server 960 may generate a tissue sample of one or more subjects 911, a tissue sample slide (pathology slide), digitized images of a tissue sample slide (pathology slide), or any combination thereof, or otherwise obtain the same from another device. Also, the user terminals 922 and 923, the image management system 930, the AI-based biomarker analysis system 940, the laboratory information management system 950, and/or the hospital or laboratory server 960 may obtain any combination of subject-specific information, such as the age, medical history, cancer treatment history, family history, past biopsy records, or disease information of the subject 911.

The scanner 921, the user terminals 922 and 923, the AI-based biomarker analysis system 940, the laboratory information management system 950, and/or the hospital or laboratory server 960 may transmit the digitized slide images, the subject-specific information, and/or results of analyzing the digitized slide images to the image management system 930 through the network 970. The image management system 930 may include a storage for storing received images and a storage for storing analysis results.

In addition, according to various embodiments of the disclosure, a machine learning model trained to predict at least one of information about at least one cell, information about at least one region, information related to a biomarker, medical diagnosis information, and/or medical treatment information from the slide image of the subject 911 may be stored in and operated by the user terminals 922 and 923, the image management system 930, or the like.

As described above, difficulty of a user in performing analysis by using raw data due to difficulty in accessing the raw data produced as a result of s pre-performed research (e.g., a clinical trial, or the like) may be resolved. In other words, the user may perform various analyses based on virtual data (i.e., data in which pre-generated survival data is reconstructed) generated by the processor 110.

In addition, as the processor 110 generates control group data to which the concept of hypothetical analysis is applied and compares and analyzes experimental group data and the control group data, validity of a biomarker identified by the machine learning model (i.e., validity in selecting responders) may be confirmed. Also, a possibility of success of an anticancer clinical trial using the machine learning model may be increased based on results output by the processor 110 (i.e., results of comparing the control group data and the experimental group data). Accordingly, a regime optimized for a patient may be provided.

Meanwhile, the above-described methods may be written as a program executable on a computer, and may be implemented in a general-purpose digital computer operating a program using a computer-readable recording medium. In addition, a structure of data used in the above-described methods may be recorded on a computer-readable medium through various methods. Examples of the computer-readable medium include storage media such as magnetic storage media (for example, read-only memory (ROM), random-access memory (RAM), universal serial bus (USB), floppy disks, and hard disks), and optical readable media (for example, CD-ROM and DVD).

One of ordinary skill in the art will understand that the disclosure may be implemented in a modified form without departing from the essential features of the disclosure. Therefore, the disclosed methods should be considered from an explanatory perspective rather than a limited perspective, and the scope of rights is exhibited in the claims, not in the above description, and should be interpreted to include all differences within the equivalent scope.

What is claimed is:

1. A computing device comprising:
a memory storing at least one program; and
a processor configured to perform at least one operation by executing the at least one program,
wherein the processor is configured to generate virtual data including information about survival rates of virtual patients included in a first group, based on pre-generated survival data, generate control group data by classifying each of the virtual patients as a responder or a non-responder according to a certain criterion, generate experimental group data based on at least one of medical images and survival data of actual patients included in a second group to which a specific regime has been applied, and output a result of comparison between the control group data and the experimental group data.

2. The computing device of claim 1, wherein the processor is further configured to generate data on at least one of progression-free survival and overall survival of each of the virtual patients by using the pre-generated survival data.

3. The computing device of claim 1, wherein the pre-generated survival data comprises a Kaplan-Meier curve.

4. The computing device of claim 3, wherein the processor is further configured to obtain the Kaplan-Meier curve for at least one of progression-free survival and overall survival, select a certain number of points on the Kaplan-Meier curve, and generate data for at least one of the progression-free survival and the overall survival of each of the virtual patients by using coordinate values corresponding to the points.

5. The computing device of claim 1, wherein the processor is further configured to determine a proportion of responders according to at least one parameter value set based on a hypothesis, and generate at least one set in which the virtual patients are classified as responders or non-responders, based on the proportion.

6. The computing device of claim 5, wherein the at least one parameter value comprises at least one of a hazard ratio of progression-free survival and a hazard ratio of overall survival.

7. The computing device of claim 5, wherein the processor is further configured to determine the proportion of responders based on information about a regime corresponding a drug that is basis of the pre-generated survival data, and generate the at least one set such that at least one parameter value is satisfied.

8. The computing device of claim 1, wherein the processor is further configured to generates at least one set in which the actual patients included in the second group are classified as responders or non-responders, based on biomarkers identified from the medical images.

9. The computing device of claim 1, wherein the processor is further configured to generates the result of comparison by comparing at least one set included in the control group data with at least one set included in the experimental group data.

10. A method of analyzing a biomarker, the method comprising:
generating virtual data including information about survival rates of virtual patients included in a first group, based on pre-generated survival data;
generating control group data by classifying each of the virtual patients as a responder or a non-responder, according to a certain criterion;
generating experimental group data based on at least one of medical images and survival data of actual patients included in a second group to which a specific regime has been applied; and
outputting a result of comparison between the control group data and the experimental group data.

11. The method of claim 10, wherein the generating of the virtual data comprises generating data on at least one of progression-free survival and overall survival of each of the virtual patients by using the pre-generated survival data.

12. The method of claim 10, wherein the pre-generated survival data comprises a Kaplan-Meier curve.

13. The method of claim 12, wherein the generating of the virtual data comprises:
obtaining the Kaplan-Meier curve for at least one of progression-free survival and overall survival, from the pre-generated survival data;
selecting a certain number of points on the Kaplan-Meier curve; and
generating data on at least one of progression-free survival and overall survival of each of the virtual patients by using coordinate values corresponding to the points.

14. The method of claim 10, wherein the generating of the control group data comprises:
determining a proportion of responders according to at least one parameter value set based on a hypothesis; and
generating at least one set in which the virtual patients are classified as responders or non-responders, based on the determined proportion.

15. The method of claim 14, wherein the at least one parameter value comprises at least one of a hazard ratio of progression-free survival and a hazard ratio of overall survival.

16. The method of claim 14, wherein the determining of the proportion of responders comprises determining the proportion of responders based on information about a regime corresponding to a drug that is basis of the pre-generated survival data, and
the generating of the at least one set comprises generating the at least one set such that the at least one parameter value is satisfied.

17. The method of claim 10, wherein the generating of the experimental group data comprises generating at least one set in which the actual patients included in the second group are classified as responders or non-responders, based on biomarkers identified from the medical images.

18. The method of claim 10, wherein the outputting comprises generating the result of comparison by comparing at least one set included in the control group data with at least one set included in the experimental group data.

19. A computer-readable recording medium having recorded thereon a program for executing, on a computer, the method of claim 10.
